(19) United States
(12) Patent Application Publication
(10) Pub. No.: US 2005/0182403 A1
(43) Pub. Date: Aug. 18, 2005

(54) Fixture for spines

(75) Inventors: Yan Wang, Beijing (CN); Cheng-Kung Cheng, Taipei (TW)

Correspondence Address: Cooper & Dunham LLP, 1185 Avenue of the Americas, New York, NY, 10036, US

(73) Assignee: Wellong Instruments Co., Ltd.

(21) Appl. No.: 10/771,601

(22) Filed: Feb. 4, 2004

(51) Int. Cl. A61B 17/56

(52) U.S. Cl. 606/69

(57) ABSTRACT

A spine fixture includes a first engaging plate having at least two first holes, a second engaging plate interconnected with the first engaging plate and having at least two second holes and screws extending to the first holes and the second holes for securely engaging the first engaging plate and the second engaging plate onto a spine.

### BACKGROUND OF THE INVENTION

1. Field of the Invention

The present invention relates to a fixture, and more particularly to a fixture for a damaged spine such that the fixture is able to fix the damaged spine in position to prevent the damaged spine from deterioration.

2. Description of Related Art

The spine is the most important bone in vertebrates as it supports the body weight to enable the animals to walk or stand upright. That is, if the spine is damaged, the vertebrate is not able to walk or stand upright comfortably anymore, and may even become paraplegic. However, should there be an accident and the spine is damaged, a conventional spinal fixture (50) as shown in FIG. 4 is introduced to the market to help stabilize the damaged spine. The conventional spine fixture has a base (51), two holes (52) and multiple elongated holes (53). In this conventional spine fixture (50), there are four elongated holes (53), wherein two elongated holes (53) are in alignment with one of the holes (52) and the other two elongated holes (53) are in alignment with the other one of the holes (52). When the conventional spine fixture (50) is in application, the technician uses bolts (not shown) to extend through either one of the two holes (52) and one or two elongated holes (53) depending on the location of the damage. Due to the elongated holes (53), after the bolts are extended through the elongated holes (53), the positioning effect of the base (51) is not as good as expected in that the bolt may slide along the elongated holes (53) such that after a long period of time using the conventional spine fixture (50), the technician has to relocate the base (51) to secure the spine again.

To overcome the shortcomings, the present invention tends to provide an improved fixture for a spine to mitigate the aforementioned problems.

### SUMMARY OF THE INVENTION

The primary objective of the present invention is to provide an improved fixture for a spine. The fixture has two engaging plates each having holes defined therethrough and reinforced ribs extending between the two engaging plates. With the structure of the fixture, the positioning effect to the damaged spine is enhanced.

Another objective of the present invention is that each reinforced rib has an extension extending into the spine such that the fixture is able to remain stably seated onto the spine.

Other objects, advantages and novel features of the invention will become more apparent from the following detailed description when taken in conjunction with the accompanying drawings.

### BRIEF DESCRIPTION OF THE DRAWINGS

FIG. 1 is an exploded perspective view of the fixture of the present invention;

FIG. 2 is an exploded perspective view of another embodiment of the fixture of the present invention;

FIG. 3 is a schematic view showing the application of the fixture of the present invention; and

FIG. 4 is a top plan view of a conventional spine fixture.

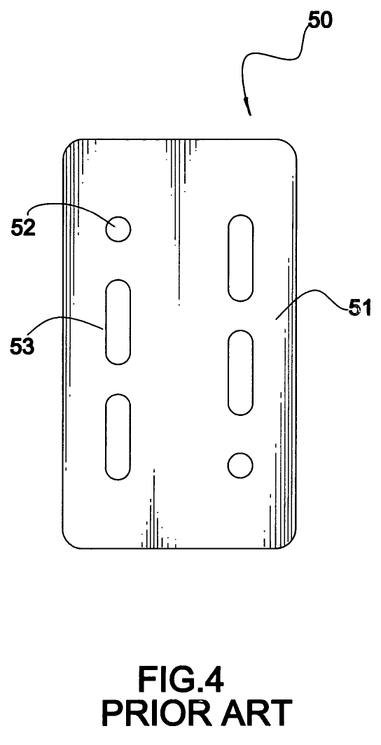

### DETAILED DESCRIPTION OF THE PREFERRED EMBODIMENT

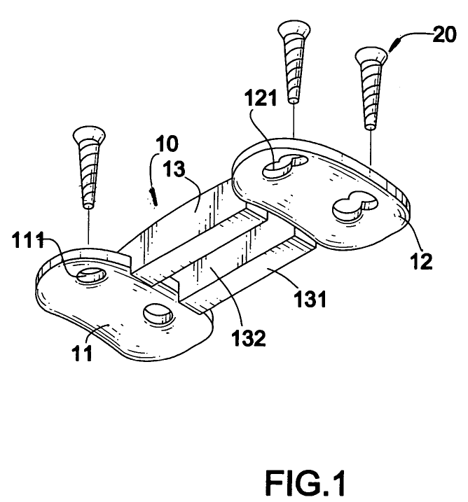

With reference to FIG. 1, the fixture (10) in accordance with the present invention has a first engaging plate (11), a second engaging plate (12) and reinforcing ribs (13) interconnecting the two engaging plates (11,12).

The first engaging plate (11) has at least two first holes (111) (two are shown in this embodiment). The second engaging plate (12) has at least two second holes (121) (two are shown in this embodiment). Each second hole (121) has a configuration the same as a numeral eight (8). Each reinforcing rib (13) is sandwiched between the first engaging plate (11) and the second engaging plate (12) and has an extension (131) integrally formed with the reinforcing rib (13).

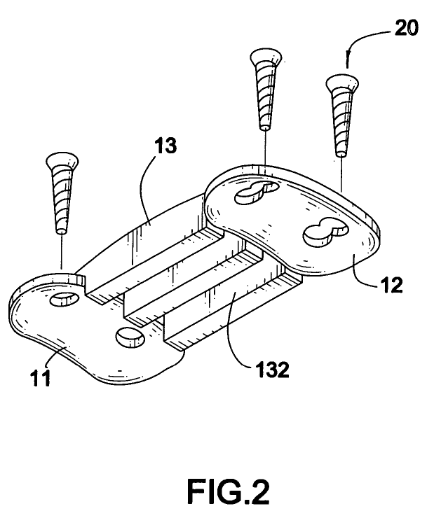

With reference to FIG. 2, it is noted that there are three reinforcing ribs (13) formed between the first engaging plate (11) and the second engaging plate (12). Screws (20) are applied to correspond to the first holes (111) and the second holes (121) in both embodiments.

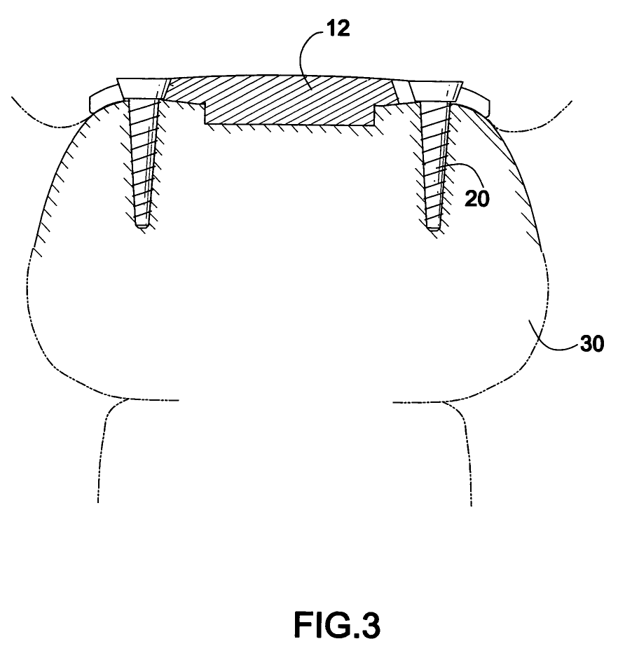

With reference to FIG. 3, when the fixture of the present invention is in application, the operator is able to use the screws (20) to extend through at least one of the first holes (111) and both of the two second holes (121) depending on the location of the damage of the spine (30). A gap (132) defined between the two adjacent reinforcing ribs (13) is provided to allow the technician to have access to the cartilage in the spine such that the technician is able to remove the cartilage partially and apply medical powder to treat the damaged spine. It may be understood that there may be provided with a through hole (not shown) in a bottom defining the gap (132) so that the medical powder applied to the damaged spine can engage with the spine directly. Due to the shape of the second holes (121), the operator not only can adapt to the location of the damage of the spine, the technician can also secure the bolts (20) relative to the second engaging plate (12). Therefore, even after a long period of time of using the fixture of the present invention on a damaged spine, tconcern about the fixture possibly becoming loose is obviated. Furthermore, the extension (131) of each of the reinforcing ribs (13) is able to be seated in a groove defined in the spine to enhance the engagement of the fixture to the spine (30).

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

1. A spine fixture comprising:
a first engaging plate having at least two first holes;
a second engaging plate interconnected with the first engaging plate and having at least two second holes; and
screws extending to the first holes and the second holes for securely engaging the first engaging plate and the second engaging plate onto a spine.

2. The fixture as claimed in claim 1, wherein reinforcing ribs are sandwiched between the first engaging plate and the second engaging plate to securely connect the first engaging plate to the second engaging plate.

3. The fixture as claimed in claim 2, wherein each reinforcing rib has an extension integrally formed with the reinforcing rib for extending into the spine to enhance engagement between the reinforcing ribs and the spine.

4. The fixture as claimed in claim 1, wherein each second hole has a configuration the same as a numeral of eight.

5. The fixture as claimed in claim 2, wherein each second hole has a configuration the same as a numeral of eight.

6. The fixture as claimed in claim 3, wherein each second hole has a configuration the same as a numeral of eight.
